(19) United States
(12) Patent Application Publication
(10) Pub. No.: US 2004/0009927 A1
(43) Pub. Date: Jan. 15, 2004

(54) Methods and compounds for reducing biofilm formulation

(76) Inventors: Tony Romeo, Decatur, GA (US); Debra W. Jackson, La Place, LA (US); Jerry W. Simecka, Mansfield, TX (US)

Correspondence Address: MERCHANT & GOULD PC, P.O. BOX 2903, MINNEAPOLIS, MN, 55402-0903, US

(21) Appl. No.: 10/438,255

(22) Filed: May 13, 2003

Related U.S. Application Data
(60) Provisional application No. 60/379,413, filed on May 13, 2002.

(51) Int. Cl. A61K 48/00; A61K 31/7076; A61K 31/70; A61K 31/4164

(52) U.S. Cl. 514/23; 514/46; 514/44; 514/396

(57) ABSTRACT

The invention relates to methods and compositions for modulating the biofilm formation by bacteria. In particular, the invention provides a method for reducing biofilm formation by bacteria comprising administering a control agent wherein the control agent is glucose, a glucose analogue, an adenylate cyclase inhibitor, a phosphodiesterase inhibitor, or a IIA^{Glc} dephosphorylation stimulator. The invention also provides a method for enhancing biofilm formation by bacteria comprising administering cAMP or a cAMP analogue.

### CROSS-REFERENCE TO REALTED APPLICATION

This application claims priority from U.S. provisional application No. 60/379,413, filed May 13, 2002, which is pending.

### FIELD OF INVENTION

The present invention relates to methods and compositions which modulate biofilm formation by bacteria.

### BACKGROUND

In the natural environment, bacteria predominantly exist in matrix-enclosed, sessile communities referred to as biofilms (Costerton, J. W., et al, Annu. Rev. Microbiol., 49: 711-745, 1995). Biofilms represent a distinct physiological state, designed to provide a protected environment which can enhance the bacterias' ability to survive antimicrobials and host defense mechanisms. Many chronic infections that are difficult or impossible to eliminate with conventional antibiotic therapies are known to involve biofilms. A partial list of the infections that involve biofilms includes: otitis media, prostatitis, vascular endocarditis, cystic fibrosis pneumonia, meliodosis, necrotising faciitis, osteomyelitis, peridontitis, biliary tract infection, struvite kidney stone and host of nosocomial infections.

Central carbon flux and its regulation represent key features of bacterial biofilm development. The RNA binding protein CsrA of E. coli represses biofilm formation and activates biofilm dispersal. The effect of CsrA on biofilm formation is mediated largely through its regulatory role in central carbon flux and intracellular glycogen synthesis and catabolism. The influence of CsrA is substantially greater than that of other regulators of E. coli biofilm formation, OmpR, RpoS or the Cpx two component signal transduction system.

Escherichia coli (E. coli) can use a number of different sugars and other carbon compounds as energy sources, including glucose and lactose. Glucose is the preferred substrate, and E. coli has elaborate regulation systems to repress other carbon-utilization genes when glucose is present. This effect is termed “Catabolite Repression”. Catabolite repression is mediated in large part by cyclic AMP and CRP (cAMP receptor protein or catabolite activating protein, CAP) in E. coli.

In classical catabolite repression, transport of glucose leads to dephosphorylation of IIA^{Glc} of the PTS system, which prevents this protein from activating membrane-bound adenylate cyclase (Cya). The binding of cAMP to CRP leads to the formation of a complex that interacts specifically and with high affinity to its cis-elements in the promoter regions of cAMP-regulated genes, and thereby regulates transcription. Cyclic AMP receptor protein levels also decline during catabolite repression.

cAMP-CRP complex activates the expression of certain operons involved in biofilm formation, flhDC, which encodes the activator protein for the flagellar cascade of gene expression and glgCAP, which encodes glycogen biosynthetic and degradative enzymes. However, the relative contributions of these or other genes to the observed catabolite repression remain to be determined.

Surprisingly, it has been found that glucose inhibits biofilm formation. Moreover, this inhibition appears to be mediated by decreased levels of cyclic AMP (cAMP). Thus, while the invention is not limited to any particular mechanism, it is believed that biofilm formation can be modulated by catabolite repression.

A role for the cAMP receptor protein (CRP) in biofilm formation has been identified.

Thus, surprising new methods and compounds for reducing biofilm formation have been developed, together with new uses for compounds in reducing biofilm formation.

### SUMMARY OF THE INVENTION

According to one aspect of the invention a method is provided for reducing or controlling biofilm formation by a bacterium comprising administering a control agent which inhibits cAMP and CRP interaction.

The control agent can be selected from a group consisting of: glucose, a glucose analogue, glucose catabolite, an adenylate cyclase inhibitor, a phosphodiesterase activator, and a stimulator of IIAGlc dephosphorylation.

The control agent can also be a nucleic acid specifying an antisense RNA adapted to interact with bacterial mRNA encoding CRP.

The method of reducing or controlling biofilm formation can further comprise administering an antibiotic.

The antibiotic can be selected from a group consisting of a: beta-lactam, vancomycin, bacitracin, macrolide, lincosamide, chloramphenicol, tetracycline, aminoglycoside, amphotericin, cefazolin, clindamycin, mupirocin, sulfonamide, trimethoprim, rifampicin, metronidazole, quinolone, novobiocin, polymixin, and gramicidin.

According to another aspect of the invention, a pharmaceutical composition is provided comprising a control agent which inhibits cAMP and CRP interaction and a suitable carrier.

The control agent can be selected from a group consisting of: glucose, a glucose analogue, glucose catabolite, an adenylate cyclase inhibitor, a phosphodiesterase activator, and a stimulator of IIAGlc dephosphorylation.

The pharmaceutical composition can further comprise an antibiotic.

The antibiotic can selected from a group consisting of a: beta-lactam, vancomycin, bacitracin, macrolide, lincosamide, chloramphenicol, tetracycline, aminoglycoside, amphotericin, cefazolin, clindamycin, mupirocin, sulfonamide, trimethoprim, rifampicin, metronidazole, quinolone, novobiocin, polymixin, and gramicidin.

According to yet another aspect of the present invention, a method is provided for screening the ability of a compound to act as a control agent which inhibits cAMP and CRP interaction comprising: treating bacterial cells with a compound; comparing biofilm formation by the treated bacterial cells and biofilm formation by untreated bacterial cells; and wherein a reduction in biofilm formation indicates that the compound is effective as a control agent.

The compound can inhibit adenylate cyclase activity.

The compound can stimulate dephosphorylation of IIAGlc.

The compound can inhibit IIAGlc activation of adenylate cyclase.

According to still another aspect of the present invention, a method is provided for enhancing biofilm formation by a bacterium comprising administering cAMP or a cAMP analogue which enhances cAMP and CRP interaction.

The cAMP analogue can be selected from a group consisting of: dibutyryl cAMP, 8-bromo-cAMP, Sp-cAMPS, 8-CPT cAMP, Rp-cAMPS, and Sp-5,6-DCL-cB1MPS.

### BRIEF DESCRIPTION OF THE DRAWINGS

FIG. 1 is a graph showing the effects of glucose on biofilm formation by E. coli K-12 strains, their csrA mutants (TR strains) (A,B), and related pathogens (C,D), in CFA 5 or LB medium.

FIG. 2 is a growth curve showing the growth of MG1655, its isogenic csrA mutant, TRMG1655, and their crp and cya derivatives.

FIG. 3 is a graph showing the effects of crp on specific biofilm formation by MG1655 and its isogenic csrA mutant TRMG1655.

FIG. 4. is a graph showing the effects of cya and exogenous cAMP on specific biofilm formation by MG1655 and its csrA mutant TRMG1655.

FIG. 5 is a graph showing the temporal effects of glucose on biofilm formation at 24 or 48 30 h of growth.

### DETAILED DESCRIPTION OF THE INVENTION

The invention is based on the discovery of biofilm formation modulation by catabolite repression. Based on this discovery, it is now possible to repress or enhance biofilm formation by bacteria. While the invention is not limited to any particular mechanism, it is believed that biofilm formation can be modulated by catabolite repression. According to the invention, various agents that enhance or reduce catabolite repression are useful for regulating biofilm formation by bacteria.

Agents which enhance catabolite repression can be used as control agents for the purpose of repressing or controlling biofilm formation. As used herein, a “control agent” is an agent which, when administered to a bacterial culture, causes a reduction in specific biofilm values (measured as A₆₃₀/mg protein) of at least 25%. By “administered to”, it is meant that the bacterial cells are contacted with the control agent for a time sufficient to enhance catabolite repression and consequently, to reduce biofilm formation.

While glucose is known to enhance catabolite repression in certain bacteria, it has been discovered that glucose also inhibits biofilm formation. The invention provides a new method of repressing or controlling biofilm formation by administering an effective amount of glucose to bacterial cells. Accordingly, other forms of glucose such glucose catabolites or glucose analogues which also enhance catabolite repression, can be used as control agents. A glucose catabolite, such as fructose 1,6-diphosphate, is a compound resulting from glucose breakdown during its utilization. A glucose analogue, such as a-methyl glucoside, is a compound which is structurally similar to glucose. Other glucose analogues are well known in the art.

The effective amount of glucose, a glucose analogue, a glucose catabolite, or inhibitor of glucose degradation as a control agent can be determined using standard biofilm assays as known in the art, including the assay provided in Example 1 below. For example, various amount of glucose, a glucose analogue, glucose catabolite, or inhibitor of glucose degradation is administered to a bacterial culture, and the amount of biofilm formation compared between treated cells and control cells. One of ordinary skill in the art can determine the effective amount of glucose, glucose analogue, glucose catabolite, or inhibitor of glucose degradation which will repress biofilm formation for a particular strain of bacteria with no more than routine experimentation. Effective amounts for other control agents disclosed herein can be determined similarly.

Other agents which enhance catabolite repression can also be used as control agents. It is known that catabolite repression is enhanced by low concentrations of cAMP. The present invention provides a new use for compounds which decrease cAMP levels for the purpose of repressing biofilm formation in bacteria. One category of control agents includes compounds which stimulate dephosphorylation of IIA^{Glc}, thereby preventing this protein from activating adenylate cyclase. An example of such a stimulator is a-methyl-D-glucoside which is transported through the PTS system and leads to dephosphorylation of IIA^{Glc}. Another category of control agents includes compounds which directly inhibit adenylate cyclase thereby inhibiting the production of cAMP. Examples of adenylate cyclase inhibitors include: 2′,5′-dideoxyadenosine, MDL-12,330A, and SQ 22536. A further category of control agents includes compounds which stimulate cAMP phosphodiesterase, an enzyme responsible for lowering cAMP levels. One example of a phosphodiesterase activator is imidazole. The effective amount of a compound falling within any of the above categories of control agents can be determined as described above with regard to the use of glucose and glucose related compounds.

It is known that cAMP/CRP activates certain operons which are involved in biofilm formation. The invention provides a further category of control agents comprising antisense oligonucleotide sequences directed to the mRNA sequence of CRP of the target bacteria.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an RNA transcript of that gene and, thereby, inhibits the transcription of that gene and/or the translation of that RNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleic acid sequence of a gene of interest, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or RNA transcripts, in preferred embodiments the antisense oligonucleotides correspond to N-terminal or 5′ upstream sites such as translation initiation, transcription initiation or promoter sites. In addition, 3′-untranslated regions may be targeted. In addition, the antisense is targeted, preferably, to sites in which RNA secondary structure is not expected and at which proteins are not expected to bind.

Preferably the antisense oligonucleotides will be directed to the CRP sequence of the target bacteria. The nucleotide sequence for CRP has been isolated and identified in many bacteria, for example Klebsiella pneumoniae (Gene Bank Accession No. AJ278967), Pasteurella multocida (Gene Bank Accession No. U95380), Escherichia coli (Gene Bank Accession No. AE016767), Haemophilus influenzae (Gene Bank Accession No. M77207), and, Xanthomonas campestris (Gene Bank Accession No. AF111840). Antisense oligonucleotides can be designed for particular bacteria using known CRP sequences. Alternatively, antisense oligonucleotides can also be directed to regions which are homologous between CRP of different bacteria. Homologous regions can be ascertained by one skilled in the art by comparing known CRP sequences using available DNA analysis techniques such as BLAST sequence alignment (Altschul, S. F., Gish, W., Miller, W., Meyers, E. W., and Lipman, D. J. (1990) Basic local alignment search tool. J Mol Biol 215:403-410.).

The antisense oligonucleotides may be prepared by standard methods which may be carried out manually or by an automated synthesizer. They also may be produced recombinantly by vectors.

In use, the control agent is preferably targeted or otherwise directed to the bacterial cells using known transport systems. In some instances the control agent is preferentially taken up or metabolized by the bacterial cells. In some instances it will be desirable to administer a precursor of a control agent which is converted or metabolized into an active form in or near to the bacterial cells of interest.

In some instances it will be desirable to administer a control agent together with a known antibacterial agent such as an antibiotic. In some instances control agents which repress biofilm formation are also useful for rendering the bacterial cells more susceptible to antibiotics.

The term “antibiotic” as used herein refers to any compound known to one of ordinary skill in the art that will inhibit the growth of, or kill, bacteria. The term “antibiotic” includes, but is not limited to, beta-lactams (penicillins and cephalosporins), vancomycins, bacitracins, macrolides (erythromycins), lincosamides (clindomycin), chloramphenicols, tetracyclines, aminoglycosides (gentamicins), amphotericins, cefazolins, clindamycins, mupirocins, sulfonamides and trimethoprim, rifampicins, metronidazoles, quinolones, novobiocins, polymixins, gramicidins or any salts or variants thereof. The antibiotic used will depend on the type of bacterial infection.

Where the control agent is an antisense oligonucleotide, the introduction of the oligonucleotides can be conveniently accomplished using methods known in the art, including liposome encapsulation. In some instances it will be desirable to employ liposomes targeted to the bacteria of interest.

In some instances, the bacterial infection to be treated is an infection by one or more types of Enterobacteriaceae. In some instances, the bacterial infection is an infection by one or more strains of E. coli. In some instances, the bacterial infection is an infection by E. coli O157:H7, Citrobacter freundii, Klebsiella pneumoniae, or Salmonella enterica Typhimurium. It will be appreciated that while examples of particular biofilm producing bacteria amenable to treatment with control agents have been disclosed, the methods and compounds of the present invention are useful in respect of a wide range of bacteria.

The invention also provides novel pharmaceutical compositions for the treatment of biofilm formation in vivo in a mammalian host (or patient), relating to a bacterial infection. Biofilm formation can be reduced or controlled through the administration of a control agent along with a suitable carrier at a dose which is effective for controlling biofilm formation and which is not toxic to mammalian host. Methods for determining effective dosages and toxicity are known in the art. In some instances it may be desirable to administer a control agent together with an antibiotic appropriate to the bacteria targeted. The type of antibiotic used will depend on the type of bacterial infection. Methods for identifying bacteria and selecting an appropriate antibiotic are known in the art.

The invention also provides a screening method for screening the ability of a compound to act as a control agent. To determine whether a compound is a control agent, bacterial cells are treated with the compound and the effect of the compound agent on biofilm formation in the treated cells, as compared to control cells, is determined. Biofilm formation can be measured using known biofilm assays, such as the assay described in Example 1. A dilution series of each of the test compounds can be prepared in order to determine the minimum inhibitory concentration (MIC) for each of the compounds. The approach for determining MIC is widely known in the art. Using known high throughput screening technology, one skilled in the art can identify compounds which repress or control biofilm formation with no more than routine experimentation.

The method for screening control agents can comprise identifying compounds which: interfere with cAMP and CRP binding, inhibit adenylate cyclase activity, stimulate dephosphorylation of IIA^{Glc}, interfere with activation of adenylate cyclase by phosphorylated IIA^{Glc}, or activate phosphodiesterase. The effect of agents on cAMP levels can be assayed using known radioimmunoassay techniques. Alternatively cAMP levels could also be analysed using known high pressure liquid chromatography (HPLC) techniques using perchlorate cell extracts. The effect of agents of phosphodiesterase activity can be assayed by the loss of cAMP as determined by radioimmunoassay or HPLC. The effect of agents on IIA^{Glc} phosphorylation can be determined by immunoprecipitating IIA^{Glc} from cell extracts previously incubated with ³²P in the presence or absence of the agent of interest.

The invention also provides a method of enhancing biofilm formation. Such enhancement may be desirable in situations where efficient bacterial proliferation is desired, such as in bioreactors. The method comprises reducing catabolite repression in the cultured bacteria. For example, a crp* mutant may be employed (crp* is a cAMP independent mutant of crp). Alternatively or in addition, exogenous cAMP and/or a suitable cAMP analogue may be added to the culture. Analogues of cAMP include dibutyryl cAMP, 8-bromo-cAMP, Sp-cAMPS, 8-CPT cAMP, Rp-cAMPS, Sp-5,6-DCL-cB1MPS. In a preferred embodiment, the use of 2 mM cAMP is effective for enhancing biofilm formation.

More specific description of methods, materials, and products according to the present invention appear in the following examples.

### Example 1

### Reduction of Biofilm Formation By Glucose or CRP-Disrupted E. coli Mutants

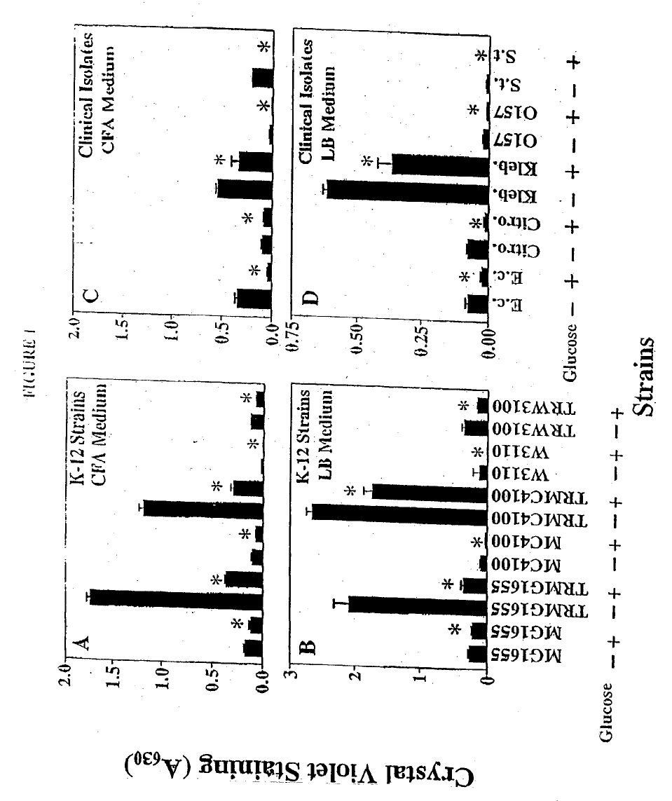

E. coli K-12 parental strains MG1655, MC41OO, W31 10 or their isogenic csrA mutants (Table 1) were grown in microtiter wells in Luria-Bertani (LB) (Ref. B) or colony forming antigen medium (CFA) (Ref. A) with or without glucose (0.2% w/v) and biofilm was quantitated after 24 h of growth using crystal violet staining (A as described (below) (FIGS. 1A and B). These and other biofilm experiments described in this manuscript were performed at least in triplicate experiments with three samples per experiment, and data were analyzed by Tukey Multigroup Analysis (Stat View-SAS Institute Inc., Cary N.C.).

FIG. 1 shows the effects of glucose on biofilm formation by E. coli K-1 2 strains, their csrA mutants (TR strains) (A,B), and related pathogens (C,D), in CFA 5 or LB medium, as indicated. Clinical strains were abbreviated as follows: E.c., E. coli P1 8; Citro., Citrobacter freundii P5; Kleb., Klebsiella pneumoniae P30; O157, E. coli O157:H7 EF302; and S.t., Salmonella enterica Typhimurium ATCC 14028. Biofilm was determined after 24 hours growth at 26° C. in the presence or absence of 0.2% glucose, as indicated. Each bar shows the average and 10 standard error of three separate experiments (P<0.0001). The * denotes significant differences with respect to cultures lacking glucose.

Glucose caused a statistically significant decrease in biofilm formation in every case, which varied from ˜30 percent reduction to ˜20-fold depending primarily on the strain background, but also on the medium. Bioflim formation by related clinical isolates, including urinary catheter isolates of E. coli, Citrobacter freundii, and Klebsiella pneumoniae, and intestinal pathogens, Salmonella enterica Typhimurium and E. coli O157:H7, was also repressed by glucose (FIGS. 1C and D). These effects generally varied from 2- to 4-fold. The three urinary catheter isolates exhibited similar repression by glucose in artificial urine medium, which mimics the urinary tract environment (data not shown).

Quantitative Biofilm Assay

Overnight cultures were inoculated 1:100 into fresh medium. In the microtiter plate assay, inoculated cultures were grown in a 96-well polystyrene microtiter plate. Growth of planktonic cells was determined by absorbance at 600 nm or total protein assay. Biofilm was measured by discarding the medium, rinsing the wells with water (three times), and staining bound cells with crystal violet (BBL) (O'Toole, et al., 1998, Mol. Microbiol. 30: 295.) The dye was solubilized with 33% acetic acid (EM Science, Gibbstown, N.J.), and absorbance at 630 nm was determined using a microtiter plate reader (DynaTech, Chantilly, Va.). For each experiment, background staining was corrected by subtracting the crystal violet bound to uninoculated controls. Comparative analyses were conducted by incubating strains within the same microtiter plate to reduce variability.

Evans, D. G., D. J. Evans Jr, and W. Tjoa. 1977. Hemagglutination of human group A erythrocytes by enterotoxigenic Escherichia coil isolated from adults with diarrhea: correlation with colonization factor. Infect. Immun. 18: 330-337.

Miller, J. H. 1972. Experiments in molecular genetics. Cold Spring Harbor Laboratory, Cold Spring Harbor, N.Y.

### Example 2

### Effect of crp and cya Deletions and Exogenous Cyclic AMP on Biofilm Formation

The effects of crp and cya deletions and exogenous cAMP on biofilm formation by MG1655 or its csrA mutant were examined. Because cAMP and CRP may have pleiotropic effects on growth, the growth curves of these strains were compared in LB (with 0.2% glucose) or CFA medium (lacking glucose) at 26° C. with shaking at 280 rpm.

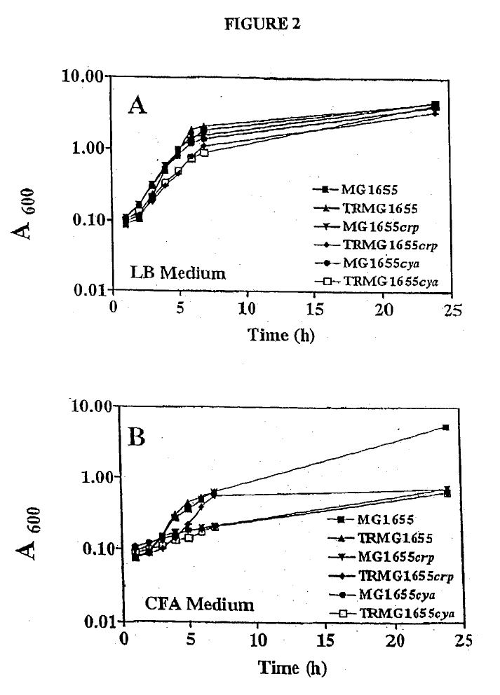

FIG. 2 shows the growth of MG1655, its isogenic csrA mutant, TRMG1655, and their crp and cya derivatives. Cultures were grown at 26° C. in LB medium containing 0.2% glucose (A) or CFA medium (B), sampled at the indicated times, and growth was determined (A₆₀₀)

Growth rates in LB medium containing 0.2% glucose were unaffected by cya and crp mutations in MG1655 and very slightly decreased in the csrA mutant background (FIG. 2A). However, all of the cya or crp mutants exhibited substantial growth defects in CFA medium (FIG. 2B). Because of these effects, biofilm formation was corrected for total cell protein to yield specific biofilm values (A₆₃₀/mg protein) in experiments with cya and crp mutants. Protein assays on cultures containing planktonic and sessile cells were conducted as described in Jackson, 2002, supra.

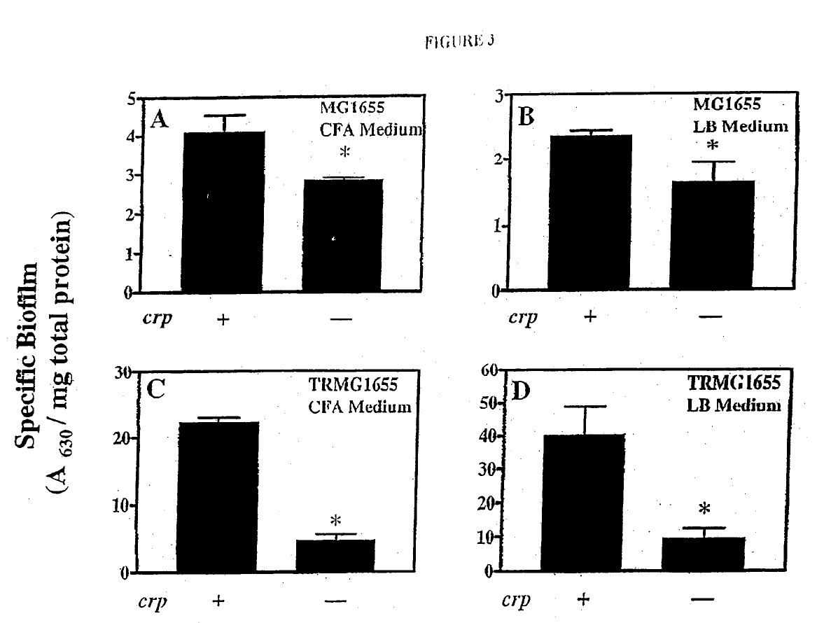

FIG. 3 shows the effects of crp on specific biofilm formation by MG1655 and its isogenic csrA mutant TRMG1655. Cultures of crp wild type or isogenic mutants were grown for 24 h in LB plus 0.2% glucose or CFA as indicated, and biofilm was determined after 24 h at 26° C. Each bar shows the average and standard error of three experiments (P<0.0001). The * indicates a significant difference between the crp mutant and its parent strain.

The disruption of crp in MG1655 or its csrA mutant significantly decreased specific biofilm formation (FIG. 3). The effect of crp was ˜30 percent in MG1655 and ˜4-fold in the csrA mutant in both media. The magnitudes of these effects were comparable to the glucose effects on these strains (FIG. 1).

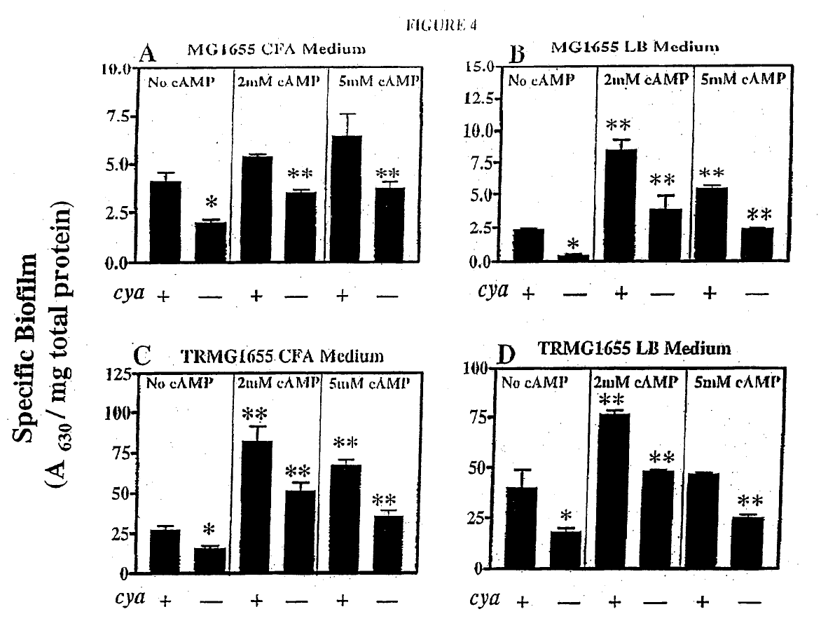

FIG. 4. shows the effects of cya and exogenous cAMP on specific biofilm formation by MG1655 and its csrA mutant TRMG1655. Cultures were grown for 24 hours in LB plus 0.2% glucose or CFA medium in the presence of 0, 2 or 5 mM cAMP, as indicated. Each bar shows the average and standard error of three experiments. The * indicates that cya disruption significantly decreased biofilm formation (P<0.0001); ** indicates that addition of cAMP to the culture resulted in a significant increase in biofilm (P<0.0001).

Disruption of cya also decreased biofilm formation in these strains (FIG. 4). The addition of cAMP (2 or 5 mM) to the growth medium of cya mutants significantly increased specific bioflim formation (˜2- to 5-fold) in all experiments, and in most cases it increased biofilm formation by cya wild type strains.

### Example 3

### Time Course of Biofilm Formation

In E. coli strains MG1655 and its csrA mutant, TRMG1655 biofilm accumulates for more than 24 hours after initial inoculation.

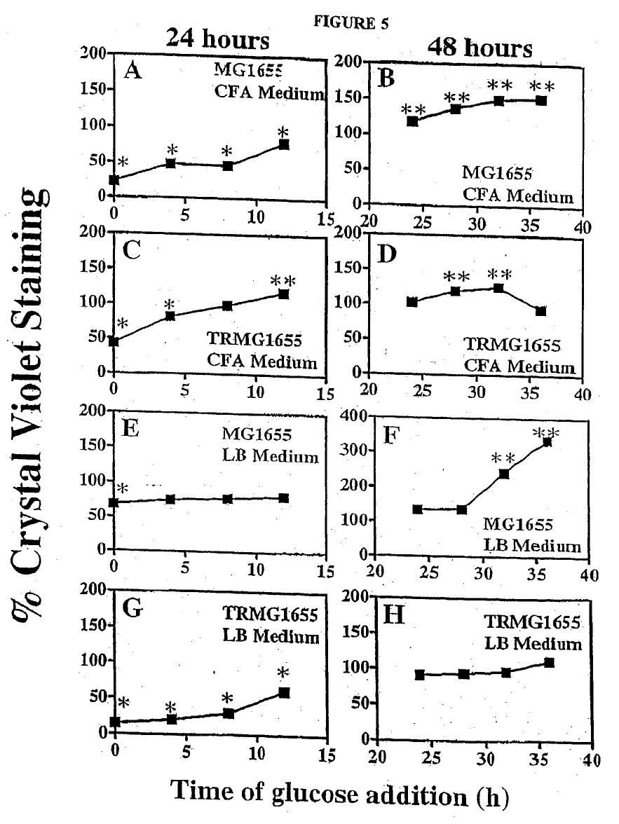

The temporal effects of catabolite repression on biofilm formed by MG1655 and its csrA mutant, TRMG1655 (FIG. 5) were examined. FIG. 5 shows the temporal effects of glucose on biofilm formation at 24 or 48 30 h of growth. Glucose (0.2% w/v final conc.) was added at the indicated times after inoculation of MG1655 or its csrA mutant, TRMG1655, into CFA or LB medium. Crystal violet staining was measured at A at either 24 or 48 h of growth, and results were calculated as % of values of the control cultures that lacked glucose. Each point shows the average and standard error of three separate experiments. The * and denote a significant decrease or increase (P<0.0001), respectively, in biofilm formation relative to controls lacking glucose.

In this experiment, 0.2% glucose (w/v final conc.) was added to cultures at various times during growth and biofilm was assayed at 24 or 48 h. The presence of glucose at the time of inoculation lead to statistically significant inhibition in every case (Panels A, C, E, and G). Thereafter, glucose effects became progressively weaker. One of the 24 h biofilms, that of TRMGI 655 in CFA medium, no longer was inhibited, but exhibited a modest yet statistically significant increase when glucose was added at 12 h (Panel C). The addition of glucose after 24 hours invariably failed to inhibit biofilm formation at 48 h. In fact, glucose addition after 24 h tended to increase biofilm formation, with the exception of TRMG1655 growing in LB medium.

TABLE 1
Bacterial Strains or phage used in this study.

| | | Reference or |
|---|---|---|
| Strains or phage | Relevant Genotype | Source |
| E.coli K-12 strains | | |
| MG1655 | F⁻λ⁻ | Michael Cashel |
| MC4100 | FΔ(argF-lac) U169rpsL relA flhD deoC ptsF rbsR | 11 |
| MLA^{a} | met gal hsdK_{R} supE supF Δcya::kanR | 10 |
| SA2777^{a} | F⁻rpsl relA Δcrp::cam | S. Garges and S. |
| | | Adhya |
| W3110 | F^{-λ}⁻mcrA mcrB IN(rrnD-rrnE)1 | Richard E. Wolf Jr. |
| TR1-5BW34114^{a} | csrA::kanR | 25 |
| Clinical Strains | | |
| P5 | Citrobacter freundii | 14 |
| P18 | Escherichia coli | 14 |
| P30 | Klebsiella pneumoniae | 14 |
| EF302 | Escherichia coil O1587:H7 | 16 |
| ATCC14028 | Salmonella enterica Typhimurium | 2 |
| Bacteriophage | | |
| P1 vir | Strictly lytic P1: forms clear plaques | 30 |

^{a}Mutant alleles Δcya, Δcrp::cam or csrA::kanR were moved among strains by P1 vir transduction or contransduction. The strain prefix TR designates the presence of csrA::kanR allele.

### REFERENCES

1. Adams, J. L., and R. J. McLean. 1999. Impact of rpoS deletion on Escherichia coli biofilms. Appl. Environ. Microbiol. 65:4285-4287.

2. Altier, C., M. Suyemoto, and S. D. Lawhon. 2000. Regulation of Salmonella enterica serovar Typhimurium invasion genes by csrA. Infect. Immun. 68:6790-6797.

3. Botsford, J. L., and J. G. Harman. 1992. Cyclic AMP in prokaryotes. Microbiol. Rev. 56:100-122.

4. Costerton, J. W., Z. Lewandowski, D. E. Caldwell, D. R. Korber, and H. M. Lappin-Scott. 1995. Microbial biofilms. Annu. Rev. Microbiol. 49:711-745.

5. Costerton, J. W., P. S. Stewart, and E. P. Greenberg. 1999. Bacterial biofilms: a common cause of persistent infections. Science 284:1318-1322.

6. Danese, P., L. A. Pratt, and R. Kolter. 2000. Exopolysaccharide production is required for development of Escherichia coli K-12 biofilm architecture. J. Bacteriol. 182:3593-3596.

7. Danese, P., L. A. Pratt, S. L. Dove, and R. Kolter. 2000. The outer membrane protein, antigen 43, mediates cell-to-cell interactions within Escherichia coli biofilms. Mol. Microbiol. 37:424-432.

8. Dorel, C., O. Vidal, C. Prigent-Combaret, I. Vallet, and P. Lejeune. 1999. Involvement of the Cpx signal transduction pathway of E. coli in biofilm formation. FEMS Microbiol. Lett. 178:169-175.

9. Evans, D. G., D. J. Evans, Jr., and W. Tjoa. 1977. Hemagglutination of human group A erythrocytes by enterotoxigenic Escherichia coli isolated from adults with diarrhea: correlation with colonization factor. Infect. Immun. 18:330-337.

10. Guerinot, M. L., and B. K. Chelm. 1984. Isolation and expression of the Bradyrhizobium japonicum adenylate cyclase gene (cya) in Escherichia coli. J. Bacteriol. 159:1068-1071.

11. Hammar, M., A. Arnquivist, Z. Bian, A. Olsen, and S. Nomark. 1995. Expression of two csg operons is required for production of fibronectin- and Congo red-binding curli polymers in Escherichia coli K-12. Mol. Microbiol. 18:661-670.

12. Ishizuka, H., A. Hanamura, T. Kunimura, and H. Aiba. 1993. A lowered concentration of cAMP receptor protein caused by glucose is an important determinant for catabolite repression in Escherichia coli. Mol. Microbiol. 10:341-350.

13. Jackson, D. W., K. Suzushi, L. Oakford, J. W. Simecka, M. E. Hart, and T. Romeo. 2002. Biofilm formation and dispersal under the influence of the global regulator CsrA of Escherichia coli. J. Bacteriol. 184:290-301.

14. Johnson, J. R., P. Delvari, and M. Azar. 1999. Activities of a nitrofurazone-containing urinary catheter and a silver hydrogel catheter against multidrug-resistant bacteria characteristic of catheter-associated urinary tract infection. Antimicrob. Agents Chemother. 43:2990-2995.

15. Kiel, K., H. Rohde, J. K. M. Knobloch, and D. Mach. 2002. Overexpression of PIA in Staphylococcus epidermidis is inhibited by inactivation of PTS glucose permease GlcA or its regulating antiterminator GlcT. Int. J. Med. Microbiol. 291(Suppl. 32.):V88.

16. Lin, J., M. P. Smith, K. C. Chapin, H. S. Baik, G. N. Bennett, and J. W. Foster. 1996. Mechanisms of acid resistance in enterohemorrhagic Escherichia coli. Appl. Environ. Microbiol. 62:3094-3100.

17. Liu, M. Y., and T. Romeo. 1997. The global regulator CsrA of Escherichia coli is a specific mRNA-binding protein. J. Bacteriol. 179:4639-4642.

18. Liu, M. Y., H. Yang, and T. Romeo. 1995. The product of the pleiotropic Escherichia coli gene csrA modulates glycogen biosynthesis via effects on mRNA stability. J. Bacteriol. 177:2663-2672.

19. Miller, J. H. 1972. Experiments in molecular genetics. Cold Spring Harbor Laboratory, Cold Spring Harbor, N.Y.

20. O'Toole, G. A., and R. Kolter. 1998. Flagellar and twitching motility are necessary for Pseudomonas aeruginosa biofilm development. Mol. Microbiol. 30:295-304.

21. O'Toole, G. A., K. A. Gibbs, P. W. Hager, P. V. Phibbs, Jr., and R. Kolter. 2000. The global carbon metabolism regulator Crc is a component of a signal transduction pathway required for biofilm development by Pseudomonas aeruginosa. J. Bacteriol. 182:425-431.

22. Pratt, L. A., and R. Kolter. 1999. Genetic analyses of bacterial biofilm formation. Curr. Opin. Microbiol. 2:598-603.

23. Pratt, L. A., and R. Kolter. 1998. Genetic analysis of Escherichia coli biofilm formation: roles of flagella, motility, chemotaxis and type I pili. Mol. Microbiol. 30:285-293.

24. Romeo, T. 1998. Global regulation by the small RNA-binding protein CsrA and the non-coding RNA molecule CsrB. Mol. Microbiol. 29:1321-1330.

25. Romeo, T., M. Gong, M. Y. Liu, and A.-M. Brun-Zinkernagel. 1993. Identification and molecular characterization of csrA, a pleiotropic gene from Escherichia coli that affects glycogen biosynthesis, gluconeogenesis, cell size, and surface properties. J. Bacteriol. 175:4744-4755.

26. Romeo, T., J. Black, and J. Preiss. 1990. Genetic regulation of glycogen synthesis in Escherichia coli: in vivo effects of the catabolite repression and stringent response systems in gig gene expression. Curr. Microbiol. 21:131-137.

27. Romeo, T., and J. Preiss. 1989. Genetic regulation of glycogen biosynthesis in Escherichia coli: in vitro effects of cyclic AMP and guanosine 5′-diphosphate 3′-diphosphate and analysis of in vivo transcripts. J. Bacteriol. 171:2773-2782.

28. Sabnis, N., H. Yang, and T. Romeo. 1995. Pleiotropic regulation of central carbohydrate metabolism in Escherichia coli via the gene csrA. J. Biol. Chem. 270:29096-29104.

29. Saier, M. H., Jr., T. M. Ramseier, and J. Reizer. 1996. Regulation of carbon utilization, p. 1325-1343. In F. C. Neidhardt, R. Curtiss III, J. L. Ingraham, E. C. C. Lin, K. B. Low, B. Magasanik, W. S. Reznikoff, M. Riley, M. Schaechter, and H. E. Umbarger (ed.), Escherichia coli and Salmonella: cellular and molecular biology, 2nd ed. ASM Press, Washington, D.C.

30. Singer, M., T. A. Baker, G. Schnitzler, S. M. Deischel, M. Goel, W. Dove, K. J. Jaacks, A. D. Grossman, J. W. Erickson, and C. A. Gross. 1989. A collection of strains containing genetically linked alternating antibiotic resistance elements for genetic mapping of Escherichia coli. Microbiol. Rev. 53:1-24.

31. Soutourina, O., A. Kolb, E. Krin, C. Laurent-Winter, S. Rimsky, A. Danchin, and P. Bertin. 1999. Multiple control of flagellum biosynthesis in Escherichia coli: role of H-NS protein and the cyclic AMP-catabolite activator protein complex in transcription of the flhDC master operon. J. Bacteriol. 181:7500-7508.

32. Tagami, H., T. Inada, T. Kunimura, and H. Aiba. 1995. Glucose lowers CRP* levels resulting in repression of the lac operon in cells lacking cAMP. Mol. Microbiol. 17:251-258.

33. Vidal, O., R. Longin, C. Prigent-Combaret, C. Dorel, M. Hooreman, and P. Lejeune. 1998. Isolation of an Escherichia coli K-12 mutant strain able to form biofilms on inert surfaces: involvement of a new ompR allele that increases curli expression. J. Bacteriol. 180:2442-2449.

34. Yang, H., M. Y. Liu, and T. Romeo. 1996. Coordinate genetic regulation of glycogen catabolism and biosynthesis in Escherichia coli via the CsrA gene product. J. Bacteriol. 178:1012-1017.

What is claimed is:

1. A method of reducing or controlling biofilm formation by a bacterium comprising administering a control agent which inhibits the interaction of cAMP and CRP or inhibits cAMP synthesis.

2. The method of claim 1 wherein the control agent is glucose.

3. The method of claim 1 wherein the control agent is a glucose analogue.

4. The method of claim 3 wherein the glucose analogue is a-methyl glucoside.

5. The method of claim 1 wherein the control agent is a glucose catabolite.

6. The method of claim 1 wherein the control agent is selected from a group consisting of: an adenylate cyclase inhibitor, a phosphodiesterase activator, and a stimulator of IIA^{Glc} dephosphorylation.

7. The method of claim 6 wherein the adenylate cyclase inhibitor is 2′5′-dideoxyadenosine.

8. The method of claim 6 wherein the phosphodiesterase activator is imidazole.

9. The method of claim 1 wherein the control agent is a nucleic acid specifying an antisense RNA adapted to interact with bacterial mRNA encoding CRP.

10. A method according to claim 1 wherein the bacterium is from the family Enterobacteriaceae.

11. A method according to claim 1 further comprising administering an antibiotic.

12. A method according to claim 11 wherein the antibiotic is selected from a group consisting of a: beta-lactam, vancomycin, bacitracin, macrolide, lincosamide, chloramphenicol, tetracycline, aminoglycoside, amphotericin, cefazolin, clindamycin, mupirocin, sulfonamide, trimethoprim, rifampicin, metronidazole, quinolone, novobiocin, polymixin, and gramicidin.

13. A pharmaceutical composition comprising a control agent which inhibits the interaction between cAMP and CRP and a suitable carrier.

14. A pharmaceutical composition according to claim 13 wherein the control agent is selected from a group consisting of: glucose, a glucose analogue, a glucose catabolite, an adenylate cyclase inhibitor, a phosphodiesterase activator and a IIA^{Glc} dephosphorylation stimulator.

15. A pharmaceutical composition according to claim 14 further comprising an antibiotic.

16. A pharmaceutical composition according claim 15 wherein the antibiotic is selected from a group consisting of a: beta-lactam, vancomycin, bacitracin, macrolide, lincosamide, chloramphenicol, tetracycline, aminoglycoside, amphotericin, cefazolin, clindamycin, mupirocin, sulfonamide, trimethoprim, rifampicin, metronidazole, quinolone, novobiocin, polymixin, and gramicidin.

17. A method for screening the ability of a compound to act as a control agent which inhibits cAMP and CRP interaction comprising:
treating bacterial cells with a compound;
comparing biofilm formation by the treated bacterial cells and biofilm formation by untreated bacterial cells;
wherein a reduction in biofilm formation indicates that the compound is effective as a control agent.

18. A method of claim 17 wherein the compound inhibits adenylate cyclase activity.

19. A method of claim 17 wherein the compound stimulates dephosphorylation of IIA^{Glc}.

20. A method of claim 17 wherein the compound inhibits IIA^{Glc} activation of adenylate cyclase.

21. A method of claim 17 wherein the compound activates cAMP phosphodiesterases.

22. A method of enhancing biofilm formation by a bacterium comprising administering cAMP or a cAMP analogue which enhances the interaction of cAMP and CRP.

23. A method of claim 19 wherein the cAMP analogue is selected from a group consisting of: dibutyryl cAMP, 8-bromo-cAMP, Sp-cAMPS, 8-CPT cAMP, Rp-cAMPS, and Sp-5,6-DCL-cB1MPS.
